(19) United States
(12) Patent Application Publication
(10) Pub. No.: US 2023/0096287 A1
(43) Pub. Date: Mar. 30, 2023

(54) FEEDTHROUGH WITH INTEGRATED ELECTRODE AND MEDICAL DEVICE

(71) Applicant: BIOTRONIK SE & Co. KG, Berlin (DE)

(72) Inventor: Martin HENSCHEL, Berlin (DE)

(73) Assignee: BIOTRONIK SE & Co. KG, Berlin (DE)

(21) Appl. No.: 17/800,767

(22) PCT Filed: Feb. 19, 2021
(86) PCT No.: PCT/EP2021/054086

§ 371 Date: Aug. 18, 2022

(30) Foreign Application Priority Data
Feb. 25, 2020 (EP) 20159226.8

(51) Int. Cl. A61N 1/375

(52) U.S. Cl. CPC A61N 1/3754; A61N 1/37518

(57) ABSTRACT

The present invention relates to an implantable medical device, comprising a housing with an electric feedthrough, wherein the electric feedthrough comprises an insulator and an electric conductor extending through the insulator, wherein insulator is joined, particularly brazed, with the electric conductor, a first electrode configured to contact a body tissue, and a second electrode configured to act as a return electrode for the first electrode, wherein the first electrode is formed by the electric conductor of the electric feedthrough and an electrode tip, wherein the electrode tip is joined, particularly welded, with the electrical conductor.

### CROSS-REFERENCE TO RELATED APPLICATIONS

This application is the United States National Phase under 35 U.S.C. § 371 of PCT International Patent Application No. PCT/EP2021/054086, filed on Feb. 19, 2021, which claims the benefit of European Patent Application No. 20159226.8, filed on Feb. 25, 2020, the disclosures of which are hereby incorporated by reference herein in their entireties.

### TECHNICAL FIELD

The present invention relates to a feedthrough with an integrated electrode and an implantable medical device comprising such feedthrough.

### BACKGROUND

In present electric medical implants, such as pacemakers or loop recorders, an electric feedthrough is commonly welded into a hermetically sealed housing. One or more feedthrough pins extending through the electric feedthrough are joined to electronic components inside the housing and to components outside the housing. In traditional pacemakers with one or more leads, those outer components, also called header components, provide an electric connection between the one or more leads with the electronic components of the implant.

However, the provision of the above described electric connection requires a relatively high number of manufacturing steps and is accordingly expensive. In addition, the common architecture of electric implants is quite space-consuming, which is particularly in newer smaller implants, such as loop recorders and intracardiac pacemakers, a significantly crucial factor.

Particularly in intracardiac pacemakers, the assembling of the header components, such as anchoring elements, is challenging and cumbersome. Known solutions incorporate multiple-axis assembly, wherein the respective components have to be precisely arranged to each other and moved. The small dimensions of the components complicate or even prevent an automated handling of the components. Accordingly, present solutions are not or limited automatable or must rely on an adhesive for assembly.

The present disclosure is directed toward overcoming one or more of the above-mentioned problems, though not necessarily limited to embodiments that do.

### SUMMARY

Accordingly, it is an objective of the present invention to provide a design of implantable medical device, in which the number of necessary connections is reduced, and which can be manufactured with significant reduced effort and costs. Particularly, it is desirable to provide a design for an intracardiac pacemaker, which enables a safe, space-saving, and automatable joinable fixation of header components, particularly anchor elements.

At least this objective is attained by an implantable medical device having the features of claim 1. Appropriate embodiments thereof are stated in the dependent claims and the following description.

According to claim 1, an implantable medical device is provided. The medical device comprises:
a housing with an electric feedthrough, wherein the electric feedthrough comprises an insulator and an electric conductor extending through the insulator, and the insulator is joined, particularly brazed, with the electric conductor,
a first electrode configured to contact a body tissue, and
a second electrode configured to act as a return electrode for the first electrode.

According to the present invention, it is particularly envisioned that the first electrode is formed by the electric conductor of the electric feedthrough and an electrode tip, wherein the electrode tip is joined, particularly welded, to the electric conductor.

Particularly, the housing and the electric feedthrough are hermetically sealed.

Particularly, the first electrode is further configured to deliver electric pulses to the body tissue and/or to sense electric pulses from the body tissue. Preferably, the first electrode is configured to both deliver electric pulses to the tissue and to sense electric pulses from the tissue.

Accordingly, the implantable medical device of the present invention is designed as a pacemaker, particularly as an intracardiac pacemaker, or as a loop recorder in one embodiment.

As described above, the first electrode if formed by two parts, one of which is the electric conductor of the electric feedthrough of the implantable medical device of the present invention, wherein the first electrode may act as pacing electrode and/or sensing electrode, e.g., in case of the medical device is designed as a intracardiac pacemaker or as a loop recorder. Advantageously, the proposed design enables a single axis assembly of the medical device of the present invention, since the first electrode is formed partly by the electric conductor of the electric feedthrough. At the same time, the first electrode may be easily adapted to requirements for electrodes contacting body tissues, e.g., by applying a coating on the electrode. This can be done by applying the coating to the electrode tip separately from the electric conductor and afterwards joining the coated electrode tip and the electric conductor in contrast to coating the whole first electrode after assembly, which would require a sophisticated masking of all parts of the implantable medical device not to be coated.

Accordingly, the electrode tip is coated in one embodiment of the implantable medical device of the present invention. In one embodiment, the electrode tip is coated with a fractal coating.

The term “fractal coating” in the context of the present specification particularly refers to a coating having a fractal or fractal-type spatial geometry. Such fractal coating has the advantage to increase the active surface (e.g., in terms of pacing and/or sensing) by from several factors to several orders of magnitude. Particularly, by increasing the electrically active surface of the first electrode by the fractal coating the medical device is able to pace and/or to sense with decreased impedances, and thereby with decreased power consumption.

Furthermore, such fractal coating is preferably applied to the electrode tip using a vacuum technology and a substantial inert material such as nitride or a noble element such as platinum, palladium or iridium. Particularly, the application of the coating material comprises the repetitive application of a defined base structure to the surface of the electrode tip and to each subsequent layer of applied material, wherein preferably in each subsequent layer, the applied defined base structure has a smaller size.

In one embodiment, the electrode tip is coated with iridium or titanium nitrite. In one embodiment, the electrode tip comprises a fractal coating with iridium and or titanium nitride. In one embodiment, the coating has a thickness in the range of 1 μm to 10 μm.

In one embodiment of the implantable medical device of the present invention, the electric conductor and/or the electrode tip is made of or comprises platinum, platinum/iridium, niobium, titanium or palladium. In particular, the electric conductor and/or the electrode tip is made of platinum-iridium alloy comprising 90 wt-% platinum and 10 wt-% iridium.

In one embodiment, the insulator is made of or comprises a ceramic or a glass. In one embodiment, the electric conductor is brazed with the insulator, preferably with gold as a solder. Ceramics and in particular Al₂O₃ are suitable as insulator material.

In one embodiment of the implantable medical device of the present invention, the electric conductor comprises an intermediate portion joined, particularly brazed, preferably with gold as a solder, to the insulator, and a distal portion protruding out of the insulator, wherein the distal portion at least partly has a larger diameter than the intermediate portion.

Advantageously, the electric conductor exhibits a wider distal “head” when compared to the rest of the electric conductor, wherein the wider distal “header” enables a better tissue contact and, thus a better pacing and/or sensing capability.

In one embodiment of the implantable medical device of the present invention, the distal portion comprises a circumferential protrusion having a larger diameter than the intermediate portion and a distal tip having a smaller diameter than the circumferential protrusion, and the electrode tip comprises a receptacle configured to receive the distal tip. In another embodiment, the distal portion comprises circumferential protrusion and a receptacle configured to receive a proximal protrusion of the electrode tip.

Advantageously, the distal tip of the electric conductor and the receptacle of the electrode tip or the receptacle of the electric conductor and the proximal protrusion of the electrode tip facilitate the alignment and/or mating of the electric conductor and the electrode tip. At the same time, the circumferential protrusion may act as a stop or locating surface for the electrode tip.

Preferably, the first electrode is designed in form of a nail, wherein the distal portion of the electric conductor and the electrode tip together form a widened nail head of the first electrode.

In one embodiment of the implantable medical device of the present invention, the electric conductor comprises a proximal portion joined, particularly soldered, to an electronic module comprised within the housing,

In one embodiment of the implantable medical device of the present invention, the proximal portion of the electrical conductor comprises a proximal tip. In one embodiment, the electronic module is joined with the electric conductor via a first terminal element having a receptacle configured to receive the proximal tip. In one embodiment, the first terminal element is solderable, i.e., comprises or essentially consists of a solderable material. Suitable solderable materials include without being restricted to copper, nickel, and alloys thereof, e.g., copper-nickel or bronze, wherein the material may be additionally coated with, e.g., palladium or tin. The first terminal element may be designed in form of a hollow cylinder, hollow disc or a solid bump. In one embodiment, the first terminal element is designed in form of a hollow cylinder made of nickel with a palladium coating or a hollow disc or a bump made of nickel that may be additionally pre-tinned.

Advantageously, the electric conductor may be more easily aligned, mated and/or joined with the electronic module by means of the proximal tip of the electric conductor or the proximal protrusion of the electrode tip.

In one embodiment of the implantable medical device of the present invention, the electric conductor, particularly the intermediate portion the electric conductor, is joined, particularly brazed or welded, to a conductor extension, wherein the conductor extension is joined, particularly soldered, to the electronic module. In one embodiment, the conductor extension is made of or comprises substantially the same material as the electric conductor. In one embodiment, the conductor extension is joined with the electronic module via a fist terminal element, wherein particularly the first terminal element is designed in form of a solid bump, preferably made of or comprising nickel that may be additionally pre-tinned. In one embodiment, the conductor extension comprises a proximal tip. In one embodiment, the conductor extension is joined to the electronic module via a first terminal element having a receptacle configured to receive the proximal tip of the conductor extension. Appropriate embodiments and examples of the first terminal element are stated above.

In one embodiment, the implantable medical device further comprises a power source, particularly a battery, comprised within the housing, wherein the electronic module is in electrical connection with the power source.

In one embodiment, the implantable medical device of the present invention further comprises a flange joined, particularly welded, to the housing, wherein the flange comprises an opening configured to receive the electrical feedthrough, and the flange has essentially the same diameter as the housing. Particularly, the insulator of the electric feedthrough is joined with the flange, preferably brazed with gold as a solder, particularly to form a hermetically sealed connection between insulator and flange.

Advantageously, such flange can be welded with the housing at an angle perpendicular to the longitudinal axis of the implantable medical device of the present invention. Thereby manufacturing of the implantable medical device is facilitated. In addition, the necessary welding spots can be distanced from critical parts of the implantable medical device, particularly the joints between electric conductor and insulator, insulator and flange, and/or between electric conductor and electronic module. Accordingly, heat introduced by welding flange and housing does not impair the integrity of the aforementioned critical parts or to a lesser degree.

In one embodiment, the implantable medical implant of the present invention comprises at least one ground contact joined, particularly welded or soldered, to the flange.

In one embodiment, the flange is joined with the at least one ground contact with a second terminal element. In one embodiment, the flange comprises a protrusion, and the at least one ground contact is joined to the protrusion via a second terminal block having a receptacle configured to receive the protrusion, wherein particularly the second terminal element is designed in form of a hollow cylinder or disc. In one embodiment, the flange comprises a receptacle configured to receive a second terminal element. In one embodiment, the receptacle of the flange is designed in form of a circumferential groove configured to receive or be mated with a matching second terminal element being designed in form of a ring. In one embodiment, the second terminal block is solderable, i.e., is made of or comprises a solderable material. Suitable solderable materials are stated above.

Advantageously, the flange may be more easily aligned, mated and/or joined with the at least one ground contact.

In another embodiment, the flange is joined, particularly welded or brazed, with a flange extension, wherein the at least one ground contact is joined with the flange via the flange extension. In one embodiment, the flange comprises a receptacle configured to receive the flange extension. The flange extension may be designed in form of a (solid) cylinder or disc. In one embodiment, the flange extension is made of or comprises a brazable material, e.g., nickel, platinum, platinum/iridium, palladium or an alloy thereof, and is particularly brazed to the flange, preferably with copper, silver, gold or an alloy thereof as a solder. In one embodiment, the flange extension is joined to the ground contact by a second terminal element, wherein particularly the second terminal element is designed in form of a solid bump.

In one embodiment of the implantable medical device of the present invention, the flange comprises a receptacle configured to receive a part of the housing. Such receptacle may be designed in form of a circumferential groove, which is configured to receive a circumferential edge of the housing. Alternatively, the receptacle may be designed in form or a circumferential edge, which is configured to receive or to be mated with a matching circumferential edge of the housing.

In one embodiment of the implantable medical device of the present invention, the housing and the flange are made of or comprises the same material. In one embodiment, the housing and the flange are made of titanium or a titanium alloy.

In one embodiment of the implantable medical device of the present invention, the second electrode is formed by a part of housing, wherein preferably at least a portion of the housing located between the first electrode and the second electrode is covered with an electrically insulating coating, e.g., a parylene.

In one embodiment, the implantable medical device of the present invention further comprises an anchor structure configured to anchor the implantable medical device in a body tissue, particularly cardiac tissue. In one embodiment, the anchor structure comprises a plurality of tines and a base ring, wherein the plurality of tines is arranged, particularly fixed, at the base ring. In one embodiment, the plurality of tines and the base ring are integrally formed in one piece.

In one embodiment of the implantable medical device of the present invention, the anchor structure is attached to the implantable medical device by a retention component. Such retention component may be designed as a header cap configured to be arranged at the distal end of the implantable device, wherein the header cap comprises a through hole, through which the first electrode extends. The header cap may be formed by two parts or may be formed in one piece.

In one embodiment of the implantable medical device of the present invention, the flange comprises a receptacle configured to receive the retention component. Particularly, the receptacle may comprise a circumferential protrusion or edge formed by the flange.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further advantages, features and embodiments of the present invention will be explained hereinafter with reference to the drawings, in which:

FIG. 1 shows a cross-section of the header section of one embodiment of the medical device of the present invention comprising a flange, a feedthrough and an electrode;

FIGS. 2 to 5 show several alternative embodiments of the header section;

FIG. 6 a top view of the header section;

FIG. 7 a bottom view of the header section, and

FIG. 8 a total view of one embodiment of the medical device of the present invention.

### DETAILED DESCRIPTION

In the present invention, a design is proposed, in which a feedthrough pin 40 for conducting electrical signals in an electric feedthrough 20 is used as an electrode of an implantable medical device 100. For this purpose, the body side 43 of the pin 40 is formed and coated such that the functions of an electrode 40, 50 can be realized. The electrode is formed by two parts 40, 50. The distal part or tip 50 of the electrode 40, 50 to be coated is welded to the feedthrough pin 40. By that, the manufacturing costs can be significantly reduced, since the distal part or tip of the electrode can be coated separately from the feedthrough pin 40. Accordingly, not the whole electric feedthrough needs to be masked for coating the electrode.

The flange 70 of the feedthrough 20 has essentially the same diameter as the implant, or more precisely the housing 10 of the implant. Thereby, the flange 10 may be welded in a radial direction with respect to the longitudinal axis of the implant 100. The welding locations 11 are thereby most distanced from the brazing or soldering locations (e.g., between pin 40 and insulator 30, between insulator 30 and flange 70 or between pin 40 and electronic module 90). In addition, a simple single axis assembly of the implant 100 is possible.

In house contacts (46, 48, 71, 74, 61, 62) of the flange 70 or the feedthrough 20 are preferably designed as being solderable. Particularly, the proximal (in house) part 46, 48 of the feedthrough pin 40 is designed as a SMT-component, which can be automatable placed on a circuit 90 (e.g., on a printed circuit board).

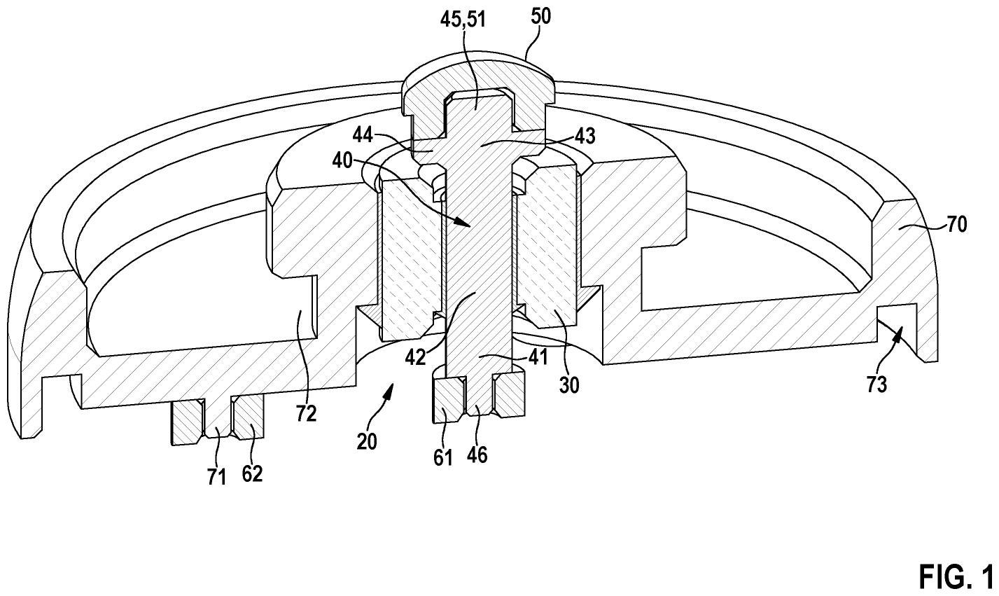

FIG. 1 shows in detail the header section of the implantable medical device 100 of the present invention with a metal flange 70, e.g., made of titanium or a titanium alloy, and an electric feedthrough 20. The electric feedthrough 20 comprises an insulator 30, e.g., made of a ceramic or glass, and a feedthrough pin 40, e.g., made of platinum, platinum/iridium, or niobium, extending through the insulator 30. Particularly, the feedthrough pin 40 is brazed with the insulator 30, and the insulator 30 is brazed with the metal flange 70, particularly with gold as a solder, in order to form a hermetic seal between pin 40 and insulator and between insulator 30 and flange 70.

The feedthrough pin 40 exhibits several distinct regions: a proximal portion 41 inside of the housing 10, an intermediate portion 42 extending through the insulator 30, and a distal portion 43 outside of the insulator 30 or the housing 10, respectively. The proximal portion 41 comprises a proximal tip 46, which is received a receptacle of a solderable first terminal element 61 configured to be soldered to the electronic module 90 of the implantable medical device 100 of the present invention. The intermediate portion 42 is at least partly brazed to the insulator 30 in order to form a hermetic seal. The distal portion 43 comprises a circumferential protrusion 44 and a distal tip 45, which is received by a receptacle 51 of a coated electrode tip 50. The circumferential protrusion 44 preferably acts as a stop or locating surface for the electrode tip 44. Feedthrough pin 40 and electrode tip 50 are welded together and form an electrode 40, 50 of the implantable medical device 100 of the present invention.

Furthermore, the metal flange 70 comprises a first receptacle 72 configured to receive a retention element of an anchor structure of the implant (not shown) and a second receptacle 73 formed as a groove and configured to receive a circumferential edge of the housing 10. The metal flange 70 further comprises a protrusion 71, which is received by a receptacle of a solderable second terminal element 62. Protrusion 71 and second terminal element 62 are configured and intended to be connected to a ground contact of the implant 100. Preferably, the first and second terminal elements are designed in form of a hollow cylinder or disc and are made of or comprise a solderable material, e.g., copper-nickel, bronze, that may be optionally coated with, e.g., palladium or tin.

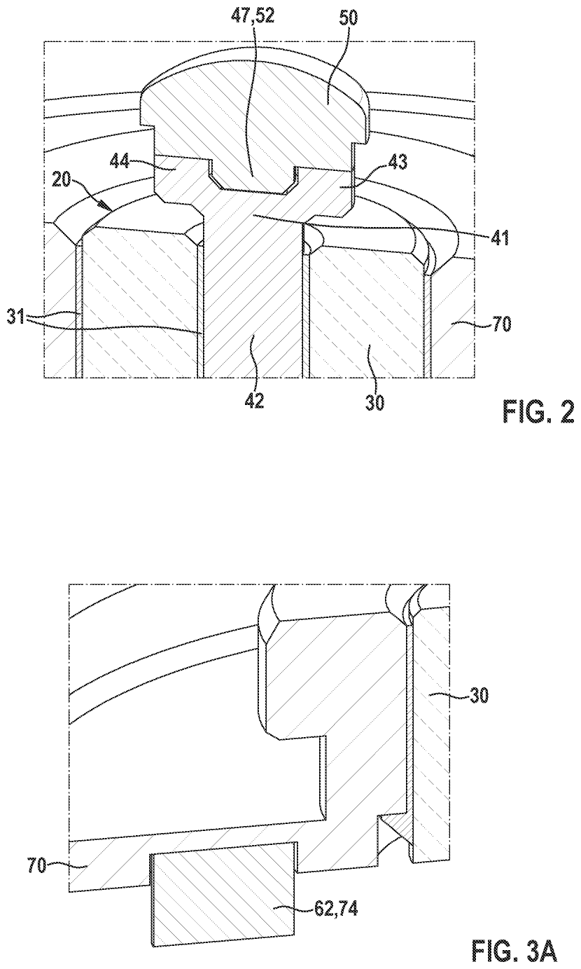

An alternative design of the feedthrough pin 40 and the electrode tip 50 is depicted in FIG. 2. There, the distal portion 43 of feedthrough pin 40 comprises a circumferential protrusion 44 as described above, but a receptacle 47, which is configured to receive a distal protrusion 52 of the electrode tip 50.

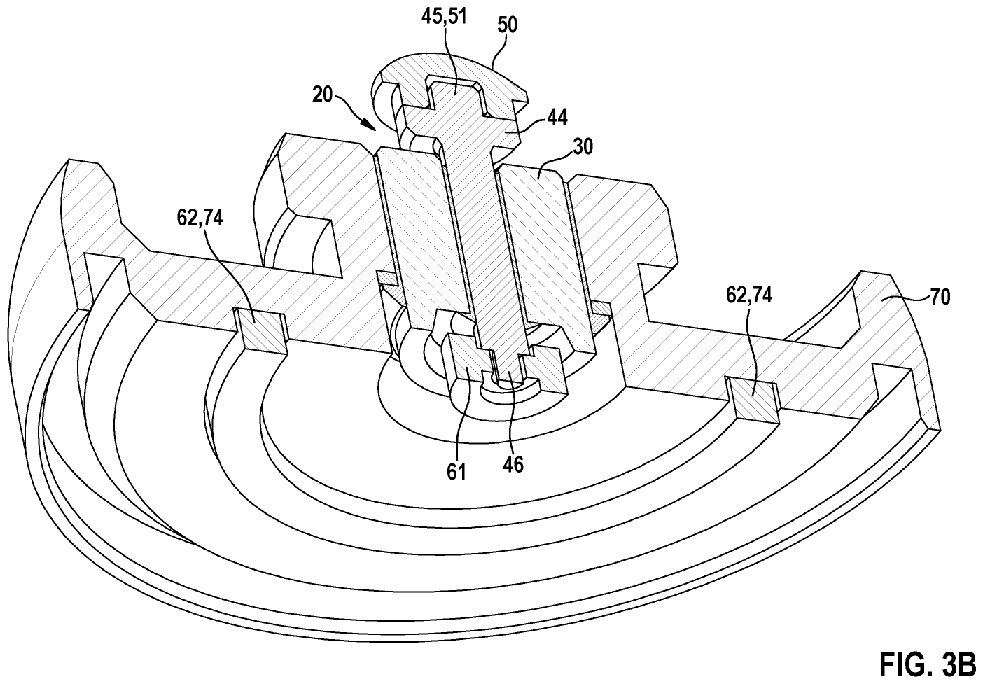

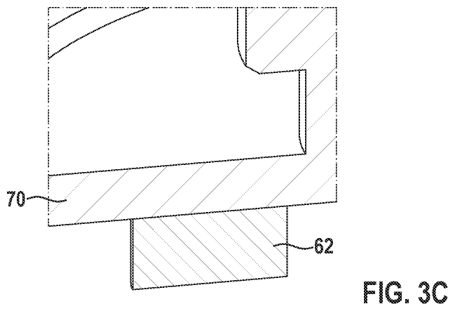

FIGS. 3A, 3B and 3C show alternative designs of the connection between flange 70 and ground contact. In one alternative design (FIGS. 3A and 3B), the flange 70 comprises a receptacle 74, which is configured to receive a second terminal element 62. In this design, the receptacle 74 is preferably designed in form of a circumferential groove, and mated with a matching second terminal element 62 being designed in form of a ring. In another alternative design, the second terminal element 62 may be joined to the inner surface of the flange 70 without a receptacle for the second terminal element 62 as depicted in FIG. 3C, wherein the second terminal element 62 is preferable designed in form of ring similar to the second terminal element of FIG. 3B. In any alternative design, the second terminal element 62 preferably is made of or comprises a solderable material as stated above.

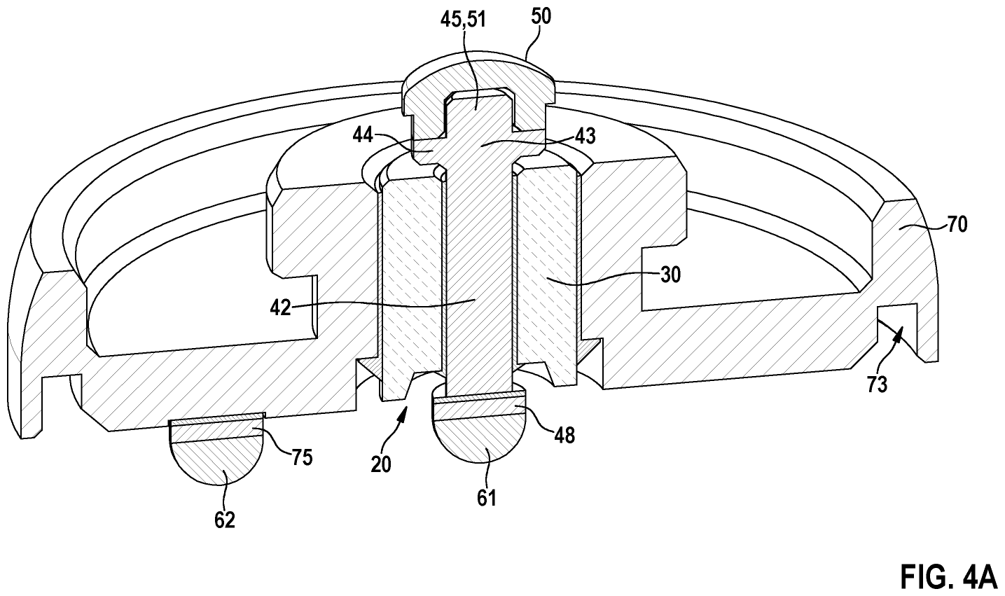

FIGS. 4A and 4B show alternative designs of the connection between flange 70 and ground contact and of the connection between the feedthrough pin 40 and the electronic module 90. There, a conductor extension 48 is brazed to the intermediate portion 42 of the feedthrough pin 40, wherein the conductor extensions 48 comprises or is joined with a first terminal element 61 being preferably designed in form of a bump (FIGS. 4A and 4B). Furthermore, a flange extension 75 is brazed with the flange 70, wherein the flange extension 75 is either arranged on the inner surface of the flange 70 as depicted in FIG. 4A or within a receptacle 74 of the flange 70 as depicted in FIG. 4B. In either case, the flange extension 75 preferably comprises or is joined with a second terminal element 62 being designed in form a bump, wherein the second terminal element 62 preferably is made of or comprises a solderable material as stated above.

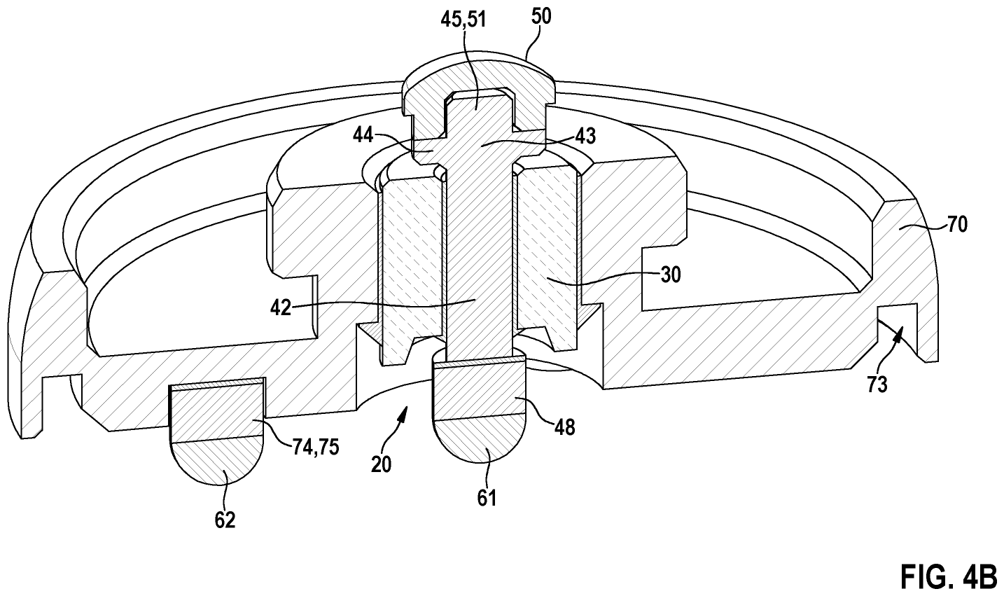

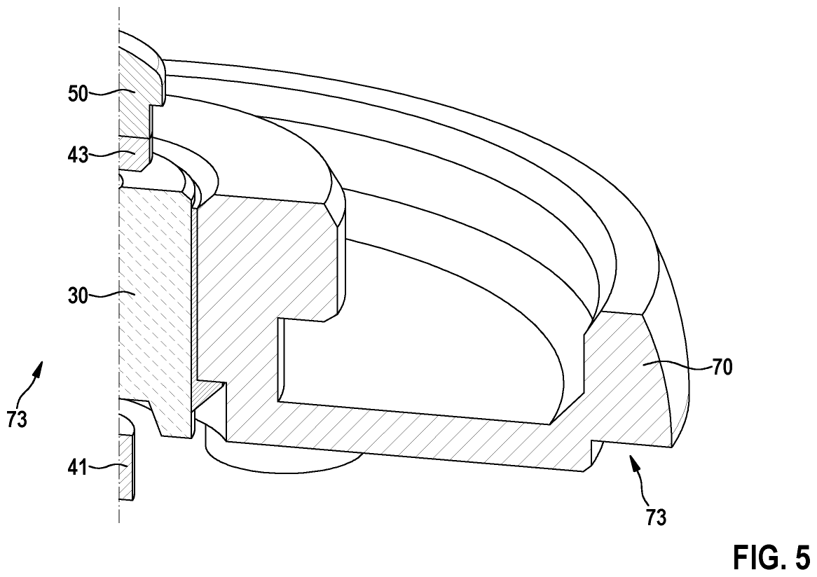

FIG. 5 shows an alternative design of the flange 70, more precisely of the receptacle 73, which is configured to receive the housing 10. In this alternative design, the receptacle 73 is designed in form of a circumferential edge configured to receive or to be mated with a matching circumferential edge of the housing 10.

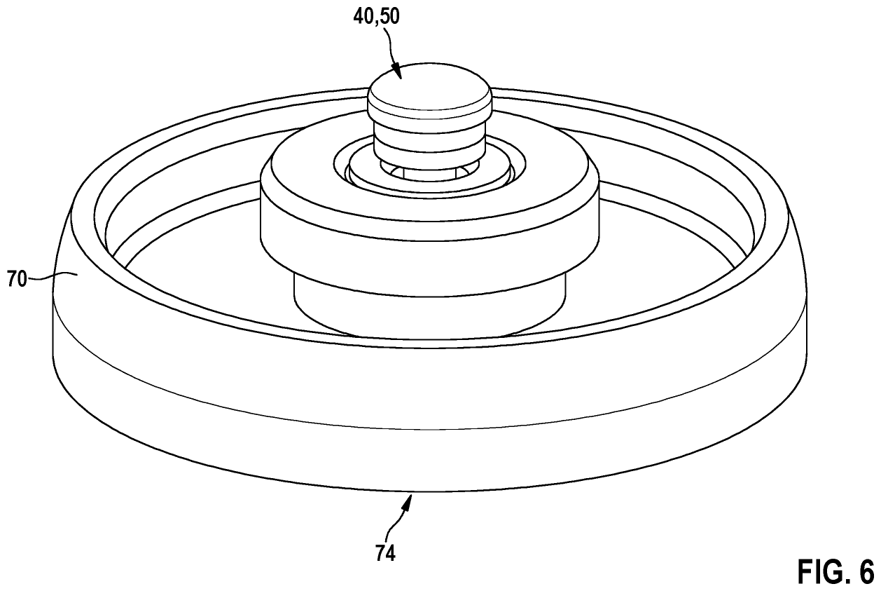

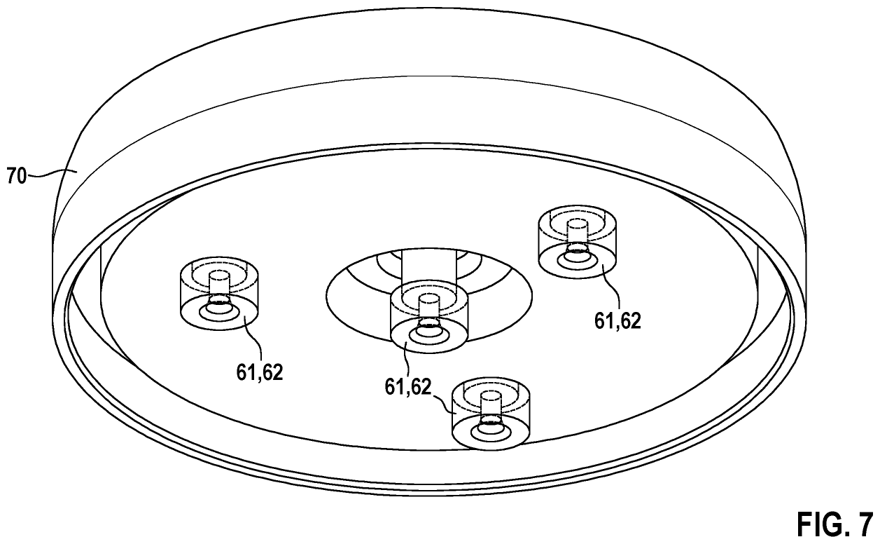

FIGS. 6 and 7 show the top side and bottom side of the above described header component of the implantable medical device 100 of the present invention. FIG. 7 shows a plurality of solderable first 61 and second terminal elements 62 configured to be soldered to the electronic module 90 and ground contacts, respectively. Preferably, each of the first 61 and second terminal elements 62 are designed in form of hollow cylinders or disc with a receptacle configured to receive the proximal tip 41 of the feedthrough pin 40 and protrusions 71 of the flange 70, respectively.

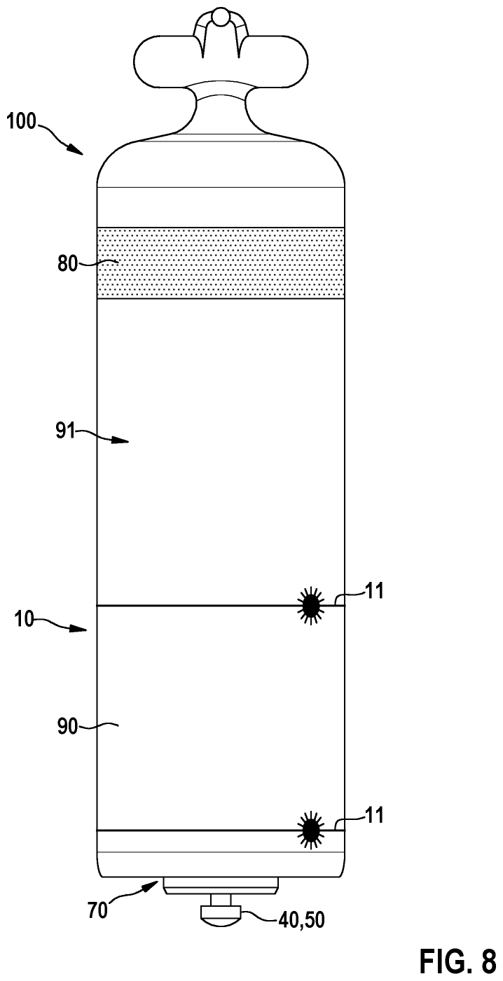

FIG. 8 show a total view of the implantable medical device 100 of the present invention. In addition to the above described header section comprising flange 70 and feedthrough 20 with feedthrough pin 40 and electrode tip 50, the implant 100 comprises a hermetically sealed housing 10. The housing 10 comprises an electronic module 90 electrically connected to the feedthrough pin 40 and a battery 91 electrically connected to the electronic module 90. Both electronic module 90 and battery 91 may be accommodated by separate housings parts, which are welded together with the flange 70 after assembly, e.g., along the welding seams 11 depicted in FIG. 8. The housing 10 further comprises a portion 80, which acts as return electrode of electrode 40, 50.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable medical device, comprising:
a housing with an electric feedthrough, wherein said electric feedthrough comprises an insulator and an electric conductor extending through said insulator, wherein insulator is joined, particularly brazed, with said electric conductor,
a first electrode configured to contact a body tissue, wherein the first electrode further is configured to deliver electric pulses to said body tissue and/or to sense electric pulses from said body tissue, and
a second electrode configured to act as a return electrode for said first electrode, wherein
said first electrode is formed by said electric conductor of said electric feedthrough and an electrode tip, wherein said electrode tip is joined, namely welded, with said electrical conductor.

2. Implantable medical device according to claim 1, wherein said electrode tip is coated with iridium or titanium nitride, wherein the coating has a thickness in the range of 1 μm to 10 μm.

3. Implantable medical device according to claim 1, wherein said electric conductor and/or said electrode tip is made of or comprises platinum, platinum/iridium, niobium, titanium or palladium.

4. Implantable medical device according to claim 1, wherein said electric conductor comprises an intermediate portion joined to said insulator, and a distal portion protruding out of said insulator, wherein said distal portion at least partly has a larger diameter than said intermediate portion.

5. Implantable medical device according to claim 4, wherein said distal portion comprises a circumferential protrusion having a larger diameter than said intermediate portion and
a distal tip having a smaller diameter than said circumferential protrusion, wherein said electrode tip comprises a receptacle configured to receive said distal tip, or
a distal receptacle configured to receive a proximal protrusion of said electrode tip.

6. Implantable medical device according to claim 1, wherein
said electric conductor comprises a proximal portion joined, namely soldered, to an electronic module comprised within said housing, and wherein said proximal portion of said electric conductor comprises a proximal tip, wherein said electronic module is joined with said electric conductor via a first terminal element having a receptacle configured to receive said proximal tip, wherein said first terminal element is solderable, or
said intermediate portion joined, namely brazed or welded, to a conductor extension, wherein said conductor extension is joined, namely soldered, to an electronic module comprised within said housing, and wherein said conductor extension comprises a proximal tip, and said electronic module is joined with said conductor extension via a first terminal element having a receptacle configured to receive said proximal tip, wherein said first terminal block is solderable.

7. Implantable medical device according to claim 1, further comprising a flange joined, namely welded, to said housing, wherein said flange comprises an opening configured to receive said electrical feedthrough, and said flange has essentially the same diameter as said housing.

8. Implantable medical device according to claim 7, wherein said medical implant comprises at least one ground contact joined, namely welded or soldered, to said flange.

9. Implantable medical device according to claim 8, wherein
said flange comprises a protrusion, and said at least one ground contact is joined to said protrusion via a second terminal element having a receptacle configured to receive said protrusion, wherein said second terminal element is solderable, or
said flange comprises a receptacle configured to receive a second terminal element, and said at least one ground contact is joined to said flange via said second terminal element, wherein said second terminal element is solderable, or
said flange is joined, namely brazed, to a flange extension, and said at least one ground contacted is joined to said flange via said flange extension and optionally a second terminal element, wherein said flange comprises a receptacle configured to receive said flange extension, or
said flange is joined with said at least one ground contact via a second terminal element, wherein said second terminal element is solderable.

10. Implantable medical device according to claim 7, wherein said flange comprises a receptacle configured to receive a part of said housing.

11. Implantable medical device according to claim 7, wherein said housing and said flange are made of or comprises the same material, namely titanium or a titanium alloy.

12. Implantable medical device according to claim 1, wherein said second electrode is formed by a part of said housing.

13. Implantable medical device according to claim1, further comprising an anchor structure configured to anchor said implantable medical in a body tissue, namely cardiac tissue, wherein said anchor structure comprises a plurality of tines and a base ring, wherein said plurality of tines is arranged, namely fixed, at said base ring, and wherein said plurality of tines and said base ring are integrally formed in one piece.

14. Implantable medical device according to claim 13, wherein said anchor structure is attached to said implantable medical device by a retention component.

15. Implantable medical device according to claim 14, wherein said flange comprises a receptacle configured to receive said retention component.
